(19) United States
(12) Patent Application Publication
(10) Pub. No.: US 2026/0294228 A1
(43) Pub. Date: Oct. 1, 2026

(54) ENDOSCOPY DEVICE HAVING ENHANCED ELECTRICAL CONTROL SYSTEM

(71) Applicant: EvoEndo, Inc., Suite 116, CO (US)

(72) Inventors: Alejandro Espinosa, Miami, FL (US); David Castano Galindo, Fort Lauderdale, FL (US)

(21) Appl. No.: 19/480,800

(22) PCT Filed: Apr. 24, 2024
(86) PCT No.: PCT/US2024/025925

§ 371 Date: Oct. 31, 2025

Related U.S. Application Data
(60) Provisional application No. 63/499,681, filed on May 2, 2023.
(60) Provisional application No. 63/499,683, filed on May 2, 2023.
(60) Provisional application No. 63/499,686, filed on May 2, 2023.

(51) Int. Cl. A61B 1/233; A61B 1/00; A61B 1/018; A61B 1/045; A61B 1/05; A61B 1/06

(52) U.S. Cl. CPC A61B 1/233; A61B 1/00009; A61B 1/00018; A61B 1/00027; A61B 1/0004; A61B 1/00045; A61B 1/00068; A61B 1/00114; A61B 1/018; A61B 1/045; A61B 1/051; A61B 1/0676

(57) ABSTRACT

An endoscope for use in a surgical procedure e.g., a pediatric unsedated trans-nasal endoscopy procedure, that includes a handle for gripping by a user and a shaft extending from the handle. The shaft may have a working channel extending longitudinally therethrough and may have a distal region configured to be inserted into a patient. The endoscope may also include a camera sensor mounted at the distal region of the shaft. The camera sensor may be configured for generating image signals related to the surgical procedure. The endoscope may also include a self-contained button assembly mounted to the handle. The self-contained button assembly may include an electrical button sub-assembly having first and second electrical buttons mounted on a circuit board. The first electrical button may be configured upon being pressed to cause a first function to be performed relative to the image signals. The second button may be configured upon being pressed to cause a second function to be performed relative to the image signals. The self-contained button assembly may also include fluid control buttons for controlling the flow of one or more fluids through a working channel of the endoscope.

### CROSS-REFERENCE TO RELATED APPLICATIONS

This application is a national stage application, filed under 35 U.S.C. § 371, of International Patent Application No. PCT/US2024/025925 filed on Apr. 24, 2024, which claims the benefit of U.S. Provisional Application Ser. No. 63/499,681, filed May 2, 2023, U.S. Provisional Application Ser. No. 63/499,683, filed May 2, 2023, and U.S. Provisional Application Ser. No. 63/499,686, filed May 2, 2023, the entire disclosures of each are incorporated herein by reference for all purposes.

### BACKGROUND

Eosinophilic esophagitis (EoE) is an increasingly common chronic inflammatory disease that affects children and adults. Because of its potential to progress to esophageal stricture and the fact that symptoms do not always correlate with degree of eosinophilia, much attention has been paid to repeated assessment of the esophageal mucosa to ensure mucosal healing following treatment. In contrast, the risks, cost and time commitment associated with traditional sedated esophagogastroduodenoscopy (EGD) can be significant and have raised concerns for providers and patients alike. To address these questions, alternative methods are needed to measure esophageal inflammation. In addition to esophagoscopy with biopsies, other technologies such as the Cytosponge, esophageal string test and confocal tethered endomicroscopy have emerged as potential alternatives for assessing mucosal inflammation.

Recent work has led to the development of trans-nasal endoscopy/esophagoscopy (TNE) to assess the esophageal mucosa in adults. In contrast to traditional EGDs, TNE offers advantages, including that it can be performed in an outpatient clinic room, requires no anesthesia or sedation, uses an adult trans-nasal gastroscope that is tolerated by adults and procures samples adequate for assessment of Barrett's Esophagus. However, the endoscopes used in the adult procedures are not appropriate for use in pediatric setting and, in fact, may be too large for many adults.

During a trans-nasal endoscopic procedure, patients may experience physical discomfort due to the endoscope being inserted into the nose, through the sinus cavities and down into the esophagus. This physical discomfort, or even the fear of being uncomfortable, can make trans-nasal endoscopy procedures mentally and emotionally distressing for a patient, too. Because it is desirable to make the procedure mentally and physically easier on the patient, it would be advantageous to optimize the endoscope being used for the procedure.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

In various embodiments, the trans-nasal endoscope addresses many challenges experienced by other systems. For example, the trans-nasal endoscope, according to various embodiments, provides a device and associated methodology that can be used to adapt TNE to assess the esophageal mucosa, gastric, and duodenal, tracheal, and bronchial mucosa in children and small adults in both a sedated and unsedated manner with a full array of steering and visualization capabilities. The trans-nasal endoscope, according to various embodiments, provides a scope that minimizes the outer diameter thereof, e.g., to reduce the discomfort to patients, while maximizing the diameter of the working channel, e.g., to provide the largest possible channel through which tools may be introduced, while simultaneously providing enhanced, e.g., four-way, steering capabilities as well as visualization functionality, as will be described more fully below. In various embodiments, the outer diameter of the endoscope shaft may be less than about 4.5 mm, and preferably is about 3.5 mm. In addition, in various embodiments, the diameter of the working channel may have a range of about 1.5 mm to 2.5 mm, and preferably is about 2.0 mm. In certain embodiments, the outer diameter of the endoscope shaft may be between about 4.3 to 4.5 mm, and the diameter of the working channel may be about 2.8 mm.

It is noted that, according to various embodiments, the endoscope described herein may be particularly well-suited for unsedated surgical procedures. Sedation is well-known, in certain circumstances, to present various risks to patients, but is often employed during surgical procedures to prevent a patient from experiencing discomfort or anxiety. By providing an endoscope having, e.g., a minimized outer diameter, a more flexible and more steerable distal regions (as will be explained in further detail below) among other advantages described below, patient discomfort and anxiety may be reduced, thereby enabling surgical procedures to be performed in an unsedated, and thus more safe, manner.

It should be recognized that, while the scope set forth herein is described for use in a trans-nasal endoscopy procedure, it may also be employed in a variety of other medical or surgical applications. For example, the scope set forth herein may be employed for use as a nasal endoscope, a trans-nasal esophagoscope, a trans-nasal gastroscope, a trans-nasal duodenoscope, a trans-nasal enteroscope, a triple endoscope, a bronchoscope, a laryngoscope, a trans-nasal gastroscope, an aerodigestive scope, and/or an endoscopic device used to visualize any body cavity into which it would fit, e.g., for examination of a stricture or the like. It should also be recognized that the endoscope described herein may be employed in fetal surgical procedures, and/or in surgical procedures that employ natural orifices, e.g., NOTES or natural orifice transluminal endoscopic procedures, such as trans-orally, trans-anally, trans-vaginally or any other natural orifice. The discussion herein of a pediatric trans-nasal endoscopy procedure is merely exemplary.

In accordance with various embodiments thereof, systems and methods are provided for use in a surgical procedure, e.g., a pediatric unsedated trans-nasal endoscopy procedure. In an embodiment, there is provided an endoscope for use in a surgical procedure that includes a handle for gripping by a user and a shaft extending from the handle. The shaft may have a working channel extending longitudinally therethrough and may have a distal region configured to be inserted into a patient. The endoscope may also include a camera sensor mounted at the distal region of the shaft. The camera sensor may be configured for generating image signals related to the surgical procedure. The endoscope may also include a self-contained button assembly mounted to the handle. The self-contained button assembly may include an electrical button sub-assembly having first and second electrical buttons mounted on a circuit board. The first electrical button may be configured upon being pressed to cause a first function to be performed relative to the image signals. The second button may be configured upon being pressed to cause a second function to be performed relative to the image signals.

In embodiments, the self-contained button assembly may also include fluid control buttons for controlling the flow of one or more fluids through the working channel. The self-contained button assembly may be mounted within the handle on a chassis which houses first, second and third fluid valves actuatable by the fluid control buttons. The first button may be configured to transmit a first signal upon being pressed, and a second button may be configured to transmit a second signal upon being pressed.

In further embodiments, the endoscope may also include a video control unit. The video control unit may include a menu of different video control functions. The video control unit may be pre-programmed such that the first signal is mapped to the first function from the menu of video control functions so that the video control unit causes the first function to be performed when the first button is pressed. The video control unit may also be pre-programmed such that the second signal is mapped to the second function from the menu of video control functions so that the video control unit causes the second function to be performed when the second button is pressed.

In still further embodiments, the endoscope may also include a video display device connected to the video control unit and configured to provide a display corresponding to the image signals generated by the camera sensor. The video control unit may cause the first or second functions to be performed by the video display device, such that the video display device changes at least one aspect of the display based on the first or second function. The menu of video control functions may include one or more of a white balancing function, an image capture function, a zoom function, a video capture start function, a video capture stop function, a brightness change function, a start video conference function, a change color spectrum function, a zoom picture function, a magnify picture function, a measure size of object function, a measure distance of object function, and a launch program or any other keyboard function on an external computer.

Still further, the endoscope may also include an illumination source at the distal region of the shaft, and an electrical cable extending longitudinally through the shaft from the illumination source and the camera at the distal region of the shaft circuit board to the handle.

In still additional embodiments, there is provided a self-contained button assembly for use in an endoscope, the endoscope being configured to generate image signals for providing an image corresponding to a surgical procedure, e.g., a pediatric unsedated trans-nasal endoscopy procedure. The self-contained button assembly may include first and second electrical buttons mounted on a circuit board. The first electrical button may be configured upon being pressed to transmit a first control signal for controlling an aspect of the image. The second electrical button may be configured upon being pressed to transmit a second control signal for controlling an aspect of the image. The self-contained button assembly may also include one or more fluid control buttons for controlling the flow of one or more fluids through the endoscope.

In embodiments, the first and second electrical buttons may be connectable to an image control unit without soldering. The first and second electrical buttons may be connectable to an image control unit. The image control unit may include at least first and second image control functions, wherein the first control signal is configured to cause the first image control function to be performed, and the second control signal is configured to cause the second image control function to be performed. The first and second image control functions may each be one image control function of a menu of different image control functions that are stored within the image control unit. The first and second control signals may be mapped to the first and second image control functions during pre-programming of the image control unit. The first and second control signals may be mapped to a menu that includes one or more of a white balancing function, an image capture function, a zoom function, a video capture start function, a video capture stop function, a brightness change function, a start video conference function, a change color spectrum function, a zoom picture function, a magnify picture function, a measure size of object function, a measure distance of object function, and a launch program or any other keyboard function on an external computer.

In various embodiments, the self-contained button assembly may be configured to transmit power from an image control unit to an illumination device located at a distal end of a shaft of the endoscope. Also, the self-contained button assembly may be configured to transmit power from an image control unit to a camera device located at a distal end of a shaft of the endoscope, the camera device configured to generate the image signals corresponding to the surgical procedure. Still further, the self-contained button assembly may be configured to transmit the image signals from the camera device to the image control unit. The image signals transmitted via the self-contained button assembly may be processable by the image control unit to generate the image on an image display device.

In still further embodiments, the self-contained button assembly may also include a valve associated with each one of the fluid control buttons, an inlet opening associated with each one of the one or more fluid control buttons, and an outlet opening connected to a working channel of the endoscope. In such embodiments, upon one of the fluid control buttons being pressed, the valve associated with that fluid button may be actuated so as to allow fluid to travel into the inlet opening associated with the fluid control button and through of the outlet opening. In embodiments, the self-contained button assembly may be mountable inside of a handle of the endoscope via a chassis within the handle.

In still further embodiments, there is provided an endoscope for use in a surgical procedure, e.g., a pediatric unsedated trans-nasal endoscopy procedure, that includes a handle for gripping by a user and a shaft extending from the handle. The shaft may have a distal region configured to be inserted into a patient. The endoscope may also include a micro-molded tip component disposed at the distal region of the shaft, the micro-molded tip component defining at least a first and a second opening. The endoscope may also have a circuit board disposed within the micro-molded tip component. The circuit board may include a camera device configured to fit within the first opening of the micro-molded tip component. The circuit board may also include an illumination device configured to fit within the second opening of the micro-molded tip component.

In embodiments, the circuit board may have at least one leg that is bent relative to the circuit board. The illumination device may be a light-emitting diode (LED) that is mounted on the at least one bent leg so as to face distally. The LED may be disposed within the second opening such that the LED is recessed relative to a distalmost face of the shaft. The circuit board may have connection points at which the camera device and the illumination device are electrically connected to wires that extend through the shaft and to the handle. The wires to which the camera device are electrically connected may be configured to provide power to the camera device and to transmit image signals from the camera device. In addition, a wire to which the illumination device is electrically connected may be configured to provide power to the illumination device. The image signals transmitted from the camera device may be transmitted via a self-contained button assembly in the handle to an image control unit for processing prior to being displayed to a user.

It should be noted, of course, that to the extent that images, e.g., image signals, image data, etc., are described herein, it will be understood that such also refers to video, e.g., video signals, video data, etc., and that the description of the image signals is intended to include single images, still images, video images, etc. without limitation.

### DRAWINGS

FIG. 1 shows a schematic representation of a trans-nasal endoscope that includes a flexible endoscope shaft, in accordance with various embodiments.

FIG. 2 is a perspective view of the handle of the trans-nasal endoscope, in accordance with various embodiments.

FIG. 3A is a front perspective view that illustrates a printed circuit board (PCB) that may be employed in the endoscope shaft, in accordance with various embodiments.

FIG. 3B is a side view of a circuit board that may be employed in the endoscope shaft, in accordance with various embodiments.

FIG. 3C is a perspective view of a mechanism for forming bends in legs of a circuit board, in accordance with various embodiments.

FIG. 4 is a front perspective view of the distal end of the endoscope shaft, in accordance with various embodiments.

FIG. 5 is a front perspective view similar to that shown in FIG. 4, but having the distal region of the endoscope shaft enclosed by a protective layer, in accordance with various embodiments.

FIG. 6 is a perspective, cut-away view of the handle that illustrates a portion of a camera electrical cable as it extends into the handle, in accordance with various embodiments.

FIG. 7A is a side view of the handle and shaft showing a strain relief mechanism, in accordance with various embodiments.

FIG. 7B is a top perspective view of the handle and shaft showing additional details of the interior of the handle, in accordance with various embodiments.

FIG. 8. is a perspective view (with various components being hidden so as not to obscure the features shown) of a self-contained button assembly mounted within the handle, in accordance with various embodiments.

FIG. 9 is a schematic view of portions of a video display system, in accordance with various embodiments.

### DETAILED DESCRIPTION

Reference will now be made in detail to specific embodiments illustrated in the accompanying drawings. In the following detailed description, numerous specific details are set forth to provide a thorough understanding. However, it will be apparent to one of ordinary skill in the art that embodiments may be practiced without these specific details. In other instances, known methods, procedures, components, circuits, and networks have not been described in detail so as not to unnecessarily obscure aspects of the embodiments.

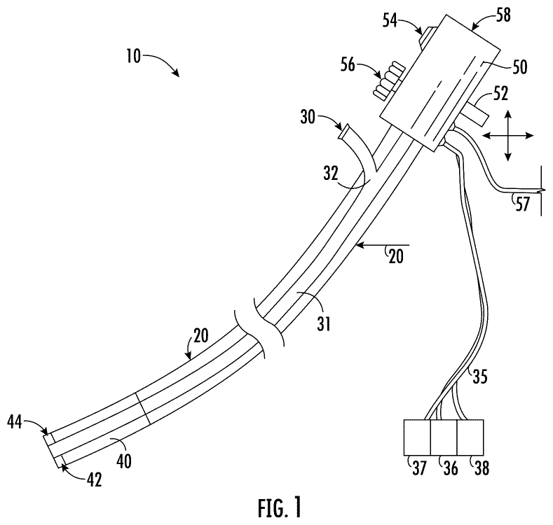

FIG. 1 is a schematic diagram of a trans-nasal endoscope 10, according to one example embodiment, illustrating some of the various features thereof. As mentioned previously, while the example embodiments set forth hereinbelow are described as an endoscope that is suitable for trans-nasal insertion into a patient, and is particularly well-suited for trans-nasal insertion into a child or small adult, it is understood that this is merely one example embodiment, and that the description hereinbelow of a trans-nasal endoscope does not preclude the use of the device in other types of procedures and for other types of patients. It should be noted that FIG. 1 is merely schematic, and thus the shape and position of the various features illustrated therein are merely exemplary. Additional figures, illustrating specific embodiments of the various features and functionality, will be provided in further detail below.

In the embodiment shown schematically in FIG. 1, the trans-nasal endoscope 10 includes a flexible endoscope shaft 20. The flexible endoscope shaft 20 has a working channel 31. The working channel 31 extends longitudinally from a distal end 40 of the endoscope shaft 20 proximally towards a handle 50 located at or near the proximal end of the trans-nasal endoscope 10. At, within or near the handle 50, the working channel 31 has a bifurcation region 32. Proximal to the bifurcation region 32, the working channel 31 splits into two channels. A first portion of the working channel 31 proximal to the bifurcation regions 32 extends towards an instrument insertion port 30 suitable for, e.g., conducting a biopsy therethrough. The instrument insertion port 30 allows an instrument, e.g., a pediatric nasal endoscope biopsy forceps or other medical device, to be inserted through the bifurcation region 32 and to, and past, the distal end 40 of the endoscope shaft 20 so as to perform a procedure, e.g., a biopsy procedure, on tissue located at or near to the distal end 40 of the endoscope shaft 20.

A second portion of the working channel 31 proximal to the bifurcation regions 32 extends towards an air, water and suction (AWS) control mechanism 52. The AWS control mechanism 52 includes various valves (not shown in this view, but shown and described in greater detail in Applicant's co-pending U.S. patent application Ser. No. 18/108,558 filed on Feb. 10, 2023, and Applicant's co-pending U.S. patent application Ser. No. 63/499,681 filed on May 2, 2023, both of which are hereby incorporated by reference herein in their entirety) that allow selective connection of the working channel 31 to the AWS tubing set 35. The AWS tubing set 35 may include one or more flexible tubes (shown and described separately and in greater detail in the above-referenced Applicant's co-pending patent applications). The AWS tubing set 35 may be connected to a water source 37 for supplying water through the working channel 31, to a suction source 36 for supplying suction through the working channel 31, and/or to an air source 38 for supplying air through the working channel 31, depending upon a user's selection via the AWS control mechanism 52. More specifically, the AWS control mechanism allows a user to direct one or more of air, suction or water through, e.g., the bifurcation region 32 and to, and past, the distal end 40 of the endoscope shaft 20 so as to enable their use during the performance of a procedure on tissue located at or near to the distal end 40 of the endoscope shaft 20.

The distal end 40 of the endoscope shaft 20 also includes an illumination source 42 to provide light at the distal end 40. In embodiments, the illumination source 42 may be connected to and at least partially controllable by an electronics control module 54 located in the handle 50. The distal end 40 of the endoscope shaft 20 also includes an image capture device 44 to convey image or video signals related to the region of the distal end 40 of the endoscope shaft 20. In embodiments, the image capture device 44 may also be connected to and at least partially controllable by the electronics control module 54 located in the handle 50. The handle 50 may also include a shaft steering mechanism 56 to control or steer the lateral displacement at the distal end 40 of the endoscope shaft 20. In addition, the handle 50 may include a video display output 57, which may be connected to and output image data to a separate image or video display or control unit (not shown in this view).

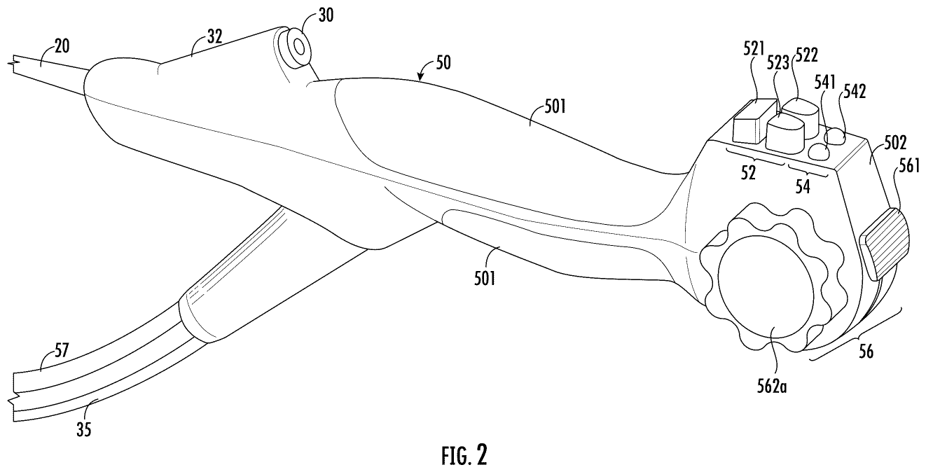

FIG. 2 is a perspective view of an example embodiment of the handle 50 of the trans-nasal endoscope 10. In this embodiment, the features and functionality that were shown schematically in FIG. 1 are provided in more detail, showing additional advantages thereof. For example, in this embodiment, the handle 50 of the trans-nasal endoscope 10 includes a gripping region 501 sized and contoured to fit comfortably in a user's hand. Located distally relative to the gripping region 501 is the bifurcation region 32. From the distalmost end of the bifurcation region 32 extends the flexible endoscope shaft 20, having a portion of the working channel 31 (not shown in this view) extending therethrough. The working channel 31 extends from the distal end 40 of the endoscope shaft 20, and splits into two channels in the bifurcation region 32. A first portion of the working channel 31 extends towards the instrument insertion port 30, which is suitable for receiving an instrument, e.g., a pediatric nasal endoscope biopsy forceps or other medical device, therethrough. The second portion of the working channel 31 proximal to the bifurcation region 32 extends proximally through the interior of the gripping region 501.

Proximal to the gripping region 501 is a control region 502 sized and shaped to extend beyond the heel of the user's hand when the palm of the user's hand is gripping the gripping region 501, enabling the control features positioned on the control region 502 to be engaged by the user's second hand when the user's first hand is gripping the gripping region 501.

In the embodiment shown in FIG. 2, the control region 502 has various control features positioned thereon. For example, the control region 502 has the AWS control mechanism 52. As set forth above, the AWS control mechanism 52 includes various features, e.g., buttons, valves, etc., that allow selective connection of the air, water and suction supply sources 36, 37, 38 to the working channel 31 via respective flexible tubes of the AWS tubing set 35. In the embodiment shown in FIG. 2, the AWS control mechanism 52 includes an air supply control button 521. The air supply control button 521 functions to selectively connect the air source 38 to the working channel 31, as is described in greater detail in the above-referenced Applicant's co-pending patent applications.

In the embodiment shown in FIG. 2, the AWS control mechanism 52 also includes a water supply control button 522. The water supply control button 522 functions to selectively connect the water source 37 to the working channel 31, as is described in greater detail in theabove-referenced Applicant's co-pending patent applications.

Still further, in the embodiment shown in FIG. 2, the AWS control mechanism 52 includes a suction supply control button 523. The suction supply control button 523 functions to selectively connect the suction source 36 to the working channel 31, as is described in greater detail in the above-referenced Applicant's co-pending patent applications.

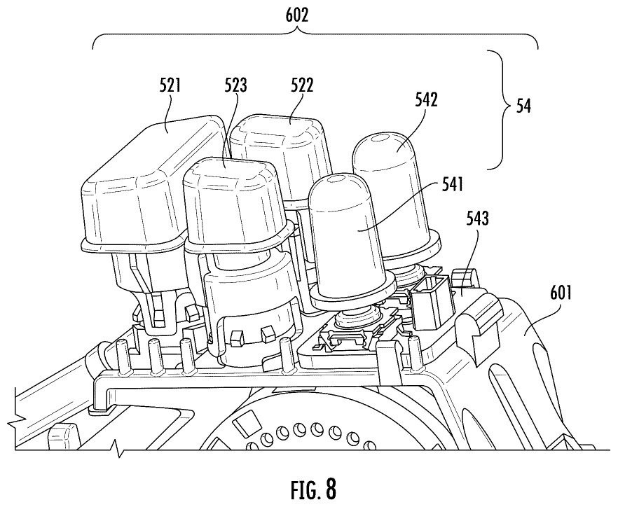

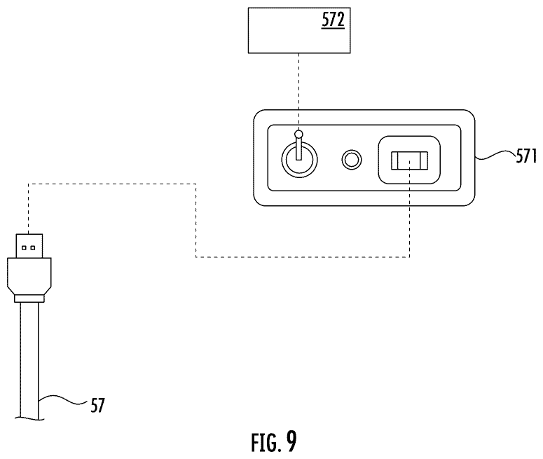

In the embodiment shown in FIG. 2, the control region 502 also has the electronics control mechanism 54. As set forth above, the electronics control mechanism 54 includes various features, e.g., buttons, electrical connections, etc., that allow selective operations related to, e. g, the image capture device 44 and/or the illumination device 44 located at the distal end 40 of the endoscope shaft 20. In the embodiment shown in FIG. 2, the electronics control mechanism 54 includes a first electrical control button 541. In this embodiment, the first electrical control button 541 may function to selectively control, e.g., a white balancing operation, by sending a corresponding signal to an image or video control unit (not shown), as is described in greater detail below in FIGS. 8 and 9.

In the embodiment shown in FIG. 2, the electronics control mechanism 54 also includes a second electrical control button 542. In this embodiment, the second electrical control button 542 may function to selectively control, e.g., the capture of image or video signals sent by the image capture device 44, e.g., such as by providing a signal to an image or video display or control unit (not shown), as is described in greater detail below in connection with FIGS. 8 and 9.

In the embodiment shown in FIG. 2, the control region 502 also has the shaft steering mechanism 56. As set forth above, the shaft steering mechanism 56 includes various features, e.g., knobs, rollers, etc., that allow a user to control or steer the lateral displacement at the distal end 40 of the endoscope shaft 20. In the embodiment shown in FIG. 2, the shaft steering mechanism 56 includes a first knob 561 for controlling a first movement of the distal end 40 of the endoscope shaft 20, as is described in greater detail in Applicant's co-pending U.S. patent application Ser. No. 18/108,562 filed on Feb. 10, 2023, and Applicant's co-pending U.S. patent application Ser. No. 63/499,683 filed on May 2, 2023, both of which are hereby incorporated by reference herein in their entirety. FIG. 2 also illustrates the shaft steering mechanism 56 including opposing roller knobs 562a, 562b (knob 562b being hidden from view in FIG. 2, but being located on the opposite side of the handle 50) for controlling additional movements of the distal end 40 of the endoscope shaft 20. Additional features and functionality of the shaft steering mechanisms 56 are shown and described in greater detail in the above-referenced Applicant's co-pending patent applications.

In addition, in the embodiment shown in FIG. 2, the handle 50 of the trans-nasal endoscope 10 includes a connection to the AWS tubing set 35. As set forth above, the AWS tubing set includes various flexible tubes that connect to the suction source 36, the water source 37 and the air source 38, as is described in greater detail in the above-referenced Applicant's co-pending patent applications. Still further, the handle 50 includes a connection to a video display output 57, e.g., for connecting to and outputting image data and/or signals to a separate video display or control unit (not shown in FIG. 2). In the embodiment shown in FIG. 2, the video display output 57 is bundled together with the AWS tubing set 35.

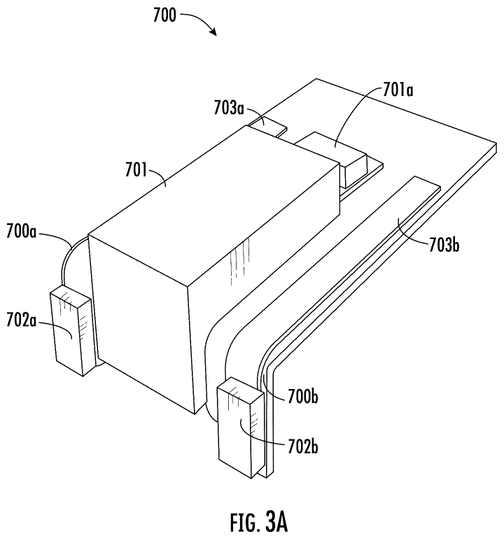

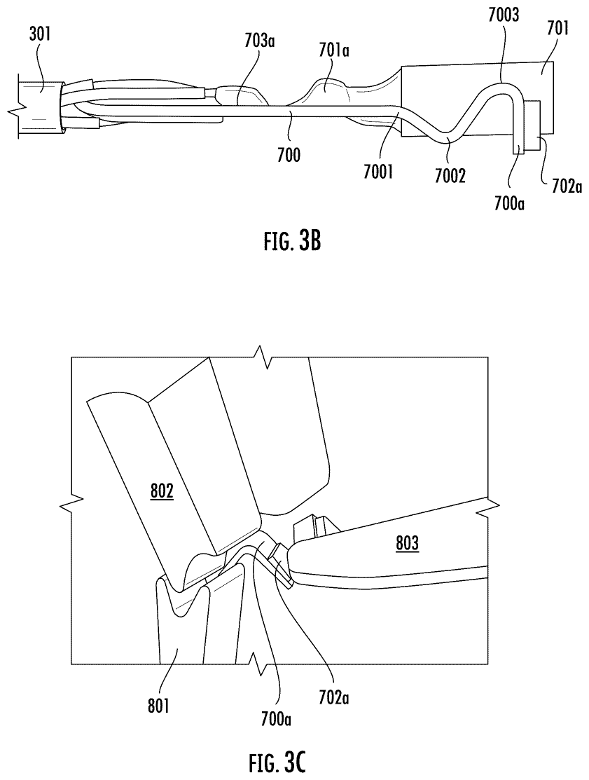

As set forth above on connection with FIG. 1, the distal end 40 of the endoscope shaft 20 may include an illumination source 42, e.g., to provide light at the distal tip 40, and an image capture device 44, e.g., to convey image or video signals related to the region of the distal end 40 of the endoscope shaft 20. FIG. 3A illustrates a front perspective view of a printed circuit board (PCB) 700 that may be employed in the endoscope shaft 20, according an embodiment. More specifically, FIG. 3A illustrates a front perspective view of a circuit board, e.g., a printed circuit board 700, that may be employed at the distal-most end 40 of the endoscope shaft 20 to provide light and generate image signals during a surgical procedure, according an embodiment.

In the embodiment shown, the printed circuit board 700 includes the imaging device 44, in the form of, e.g., a camera sensor 701. It is noted that the imaging device 44 may be any device configured to detect light reflected from the light source 42 and output an image signal. The imaging device 44 can be, for example, a charged coupled device (“CCD”) or other suitable imaging sensor. In some embodiments, the imaging device 44 may include at least two lenses providing stereo imaging, or can be an omnidirectional camera.

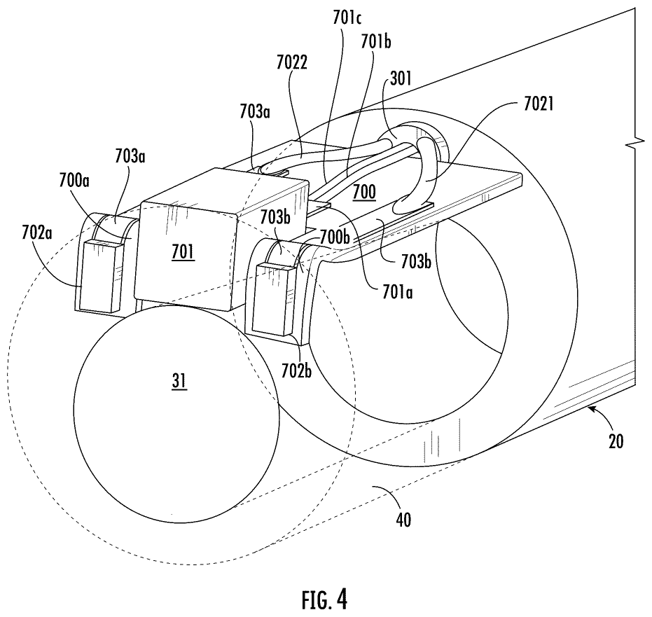

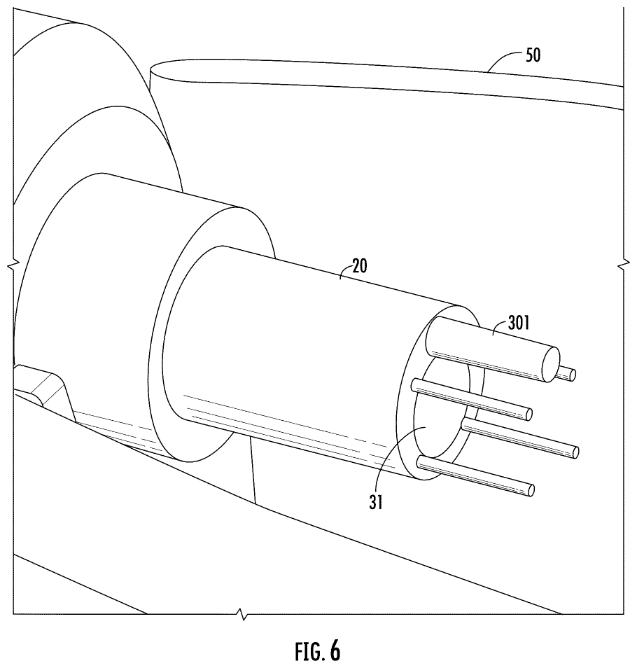

In the embodiment shown, the camera sensor 701 is located in the center of the printed circuit board 700. FIG. 3A also shows that the printed circuit board 700 includes a connection site 701a at which the camera sensor 701 may be connected to a data wire and/or power wire which may be collectively run through a cable, such as an electrical cable 301, that extends longitudinally along the endoscope shaft 20, as will be shown and described in greater detail below in connection with, e.g., FIGS. 4 and 6.

In the embodiment shown, the printed circuit board 700 also includes the illumination source 42, e.g., in this case in the form of a pair of LEDs 702a and 702b. Of course, in other embodiments, a single illumination source, e.g., a single LED may be employed. Furthermore, it should be recognized that, in other embodiments, illumination sources other than LEDs, e.g., a halogen bulb, an incandescent bulb, or other suitable light emitter may be employed. Returning to the embodiment shown in FIG. 3A, the pair of LEDs 702a, 702b may be located on opposite sides of the camera sensor 701, so as to be positioned at or near to the opposite lateral edges of the printed circuit board 700. FIG. 3A also shows that the printed circuit board 700 includes connection sites 703a, 703b at which the pair of LEDs 702a, 702b may respectively be connected to one or more power wires, which may be collectively run through a cable, such as the electrical cable 301, that extends longitudinally along the endoscope shaft 20, as will be shown and described in greater detail below in connection with FIG. 4.

Advantageously, the pair of LEDs 702a, 702b may be configured so as to face distally. In this way, the pair of LEDs 702a, 702b may provide increased illumination in a forward-facing direction, thereby primarily illuminating that region within a patient that is located directly in front of the distal end 40 of the endoscope shaft 20. In the embodiment shown, the pair of LEDs 702a, 702b is configured so as to face distally by virtue of distal leg portions 700a, 700b of the printed circuit board 700, and the pair of LEDs 702a, 702b mounted thereon, being bent downwardly, e.g., radially inwardly (in this view) at, e.g., an approximately 90 degree angle so as to be perpendicular, or generally perpendicular, relative to the proximal portion of the printed circuit board 700.

Still further, the printed circuit board 700 may be configured, in accordance with certain embodiments, such that the distal-most face of the pair of LEDs 702a, 702b is flush, e.g., equidistant in a longitudinal direction, with a distal-most face of the camera sensor 701. Having the distal-most face of the pair of LEDs 702a, 702b be flush with a distal-most face of the camera sensor 701 may provide several advantages, e.g., improving the image quality by reducing excessive glare or backlight reflection, additional details of which are set forth in Applicant's co-pending U.S. patent application Ser. No. 18/108,564 filed on Feb. 10, 2023, the entire contents of which are hereby incorporated by reference herein.

As set forth above, FIG. 3A illustrates a front perspective view of a circuit board 700 that may be employed in the endoscope shaft 20 and that includes, according an embodiment, distal leg portions 700a, 700b that are bent downwardly, e.g., radially inwardly (in this view) at, e.g., an approximately 90 degree angle so as to be perpendicular, or generally perpendicular, relative to the proximal portion of the printed circuit board 700. Other configurations for the distal leg portions 700a, 700b may also be employed. For example, FIG. 3B is a side view of a circuit board 700 that may be employed in the endoscope shaft 20 and that includes, according an embodiment, distal leg portions 700a, 700b that include multiple bent regions, e.g., bent regions 7001, 7002, 7003. Collectively, in this embodiment, the multiple bent regions, e.g., bent regions 7001, 7002, 7003 together result in a distalmost portion of each leg 700a, 700b being disposed in a generally perpendicular position relative to the proximal portion of the printed circuit board 700, such that each one of the pair of LEDs 702a, 702b are facing distally.

Of course, any number or degree of bent regions may be employed, according to various embodiments, and these bent regions may be formed by any desired configuration of equipment suitable for doing so. For example, FIG. 3C illustrates a molding process by which several molding dies, e.g., molding dies 801, 802, 803, collectively act on the distal leg portions 700a, 700b so as to bend the distal leg portions 700a, 700b into the shape shown in FIG. 3B.

FIG. 4 is a front perspective view of the distal end 40 of the endoscope shaft 20, according to an embodiment. In FIG. 4, some components of the endoscope shaft 20 are hidden or shown in phantom so as not to obscure the features shown. In the embodiment shown, the proximal end of the printed circuit board 700 may abut, or otherwise be adjacent to the electrical cable 301 extending longitudinally through the endoscope shaft 20. On the printed circuit board 700, the connection site 701a connects, e.g., by soldering thereto, the camera sensor 701 to a data wire 701b and power wire 701c which collectively run through the electrical cable 301, that extends longitudinally along the endoscope shaft 20. Likewise, on the printed circuit board 700, the connection sites 703a, 703b connect, e.g., by soldering thereto, the respective LEDs 702a, 702b to respective power wires 7021, 7022 which run through the electrical cable 301.

As set forth above, the printed circuit board 700 may be configured, in accordance with certain embodiments, such that the distal-most face of the pair of LEDs 702a, 702b is flush, e.g., equidistant in a longitudinal direction, with a distal-most face of the camera sensor 701. As shown in FIG. 4, in certain embodiments, the distal-most face of the pair of LEDs 702a, 702b and the distal-most face of the camera sensor 701 may be flush relative to each other and to the distal-most face of the endoscope shaft 20. Alternatively, and as shown in FIG. 3B, the distal-most face of the pair of LEDs 702a, 702b may be recessed relative to a distal-most face of the camera sensor 701 and/or to the distal-most face of the endoscope shaft 20. In addition to the advantages set forth above (e.g., less glare and/or backlight reflection, etc), having the distal-most face of the pair of LEDs 702a, 702b and the distal-most face of the camera sensor 701 also be flush and/or recessed relative to the distal-most face of the endoscope shaft 20 may have still further advantages. For example, having the distal-most face of the pair of LEDs 702a, 702b and the distal-most face of the camera sensor 701 also be flush and/or recessed relative to the distal-most face of the endoscope shaft 20 may simplify manufacturing, in that a protuberance-free surface is provided at the distal-most face of the endoscope shaft 20 for any protective layer, e.g., coating or laminate etc., that may be applied to such distal-most face of the endoscope shaft 20. Furthermore, having the distal-most face of the pair of LEDs 702a, 702b and the distal-most face of the camera sensor 701 also be flush and/or recessed relative to the distal-most face of the endoscope shaft 20 may simplify operation, in that a protuberance-free surface being provided at the distal-most face of the endoscope shaft 20 may help reduce the possibility that an instrument that is passed through the working channel of the endoscope shaft 20 is snagged on or otherwise undesirably contacts either of the pair of LEDs 702a, 702b or the camera sensor 701. Still further, having the distal-most face of the pair of LEDs 702a, 702b and the distal-most face of the camera sensor 701 also be flush relative to the distal-most face of the endoscope shaft 20 may help improve safety, in that a protuberance-free surface being provided at the distal-most face of the endoscope shaft 20 may help reduce the possibility that the distal-most face of the endoscope shaft 20 irritates or otherwise injures the sensitive tissue within a patient as the endoscope shaft 20 is introduced and inserted through the patient's nasal cavity, sinus cavity, esophagus, etc.

In this embodiment, the printed circuit board 700 is mounted within the shaft wall of the endoscope shaft 20. In the view shown, the printed circuit board 700 is positioned entirely within the shaft wall, e.g., such that all portions of the printed circuit board 700 are positioned laterally outside of the inner diameter of the working channel 31, while all portions of the printed circuit board 700 are positioned laterally within the outer diameter of the endoscope shaft 20. Having all portions of the printed circuit board 700 be positioned laterally outside of the inner diameter of the working channel 31, while also being positioned laterally within the outer diameter of the endoscope shaft 20, may provide several advantages. For example, having all portions of the printed circuit board 700 be positioned laterally outside of the inner diameter of the working channel 31, while also being positioned laterally within the outer diameter of the endoscope shaft 20, enables the working channel 31 to be completely clear of any obstructions. Enabling the working channel 31 to be completely clear of any obstructions may simplify manufacturing, e.g., in that any material that forms the endoscope shaft 20 or any protective layer or coating thereof may avoid the working channel 31. Furthermore, enabling the working channel 31 to be completely clear of any obstructions may simplify operation, in that an obstruction-free working channel 31 may help reduce the possibility that an instrument that is passed through the working channel 31 of the endoscope shaft 20 is snagged on or otherwise interferes with the function of, e.g., damaging or blocking the view of, the pair of LEDs 702a, 702b or the camera sensor 701 while within the working channel 31. Still further, enabling the working channel 31 to be completely clear of any obstructions may help improve safety, in that an obstruction-free working channel 31 may help reduce the possibility that an instrument inserted through the working channel 31 is damaged or otherwise impeded in its movement, which could negatively impact the surgical procedure, e.g., it could cause a biopsy sample to be harmed or dropped, it could cause broken components to be left behind in the surgical site, etc.

The configuration of the printed circuit board 700, and of the camera sensor 701 and the pair of LEDs 702a, 702b mounted thereon, as shown in FIG. 4, may also provide the advantage that the shaft wall of the endoscope shaft 20 may have the smallest thickness possible. Having the smallest wall thickness possible for the endoscope shaft 20 may help enable the endoscope shaft 20 to maximize the inner diameter of the working channel, e.g., so as to enable the largest range of instruments to be passed therethrough, while minimizing the outer diameter of the endoscope shaft 20, e.g., so as to cause as little discomfort to the patient as possible when the endoscope shaft 20 is inserted into the patient. This, in turn, may help to optimize the endoscope shaft for procedures such as, e.g., pediatric trans-nasal endoscopy procedures, for which these characteristics of the endoscope shaft 20 are particularly important.

As mentioned above, in the embodiment shown in FIG. 4, the pair of LEDs 702a, 702b is configured so as to face distally by virtue of distal leg portions 700a, 700b of the printed circuit board 700, and the pair of LEDs 702a, 702b mounted thereon, being bent downwardly, e.g., radially inwardly (in this view) at a 90 degree angle so as to be perpendicular relative to the proximal portion of the printed circuit board 700. Because the working channel 31 is round and its outer diameter thereby has a curvature, the distal legs 700a, 700b of the printed circuit board 700 are, in the embodiment shown, advantageously bent into a position such that the bottom-most edge of the LEDs 702a, 702b are positioned lower than (in this view) the bottom-most edge of the camera sensor 701. This positions the pair of LEDs 702a, 702b closer to the center of the working channel 31 than the pair of LEDs 702a, 702b would otherwise be were the distal legs 700a, 700b of the printed circuit board 700 instead bent into a position at which the bottom-most edge of the LEDs 702a, 702b were even with the bottom-most edge of the camera sensor 701. Thus, having the distal legs 700a, 700b of the printed circuit board 700 bent into a position such that the bottom-most edge of the LEDs 702a, 702b are positioned lower than the bottom-most edge of the camera sensor 701 helps to enable the wall thickness of the endoscope shaft 20 to be minimized, since this configuration follows the curvature of the working channel 31 and the outer diameter of the endoscope shaft 20. Still further, having the distal legs 700a, 700b of the printed circuit board 700 bent into a position such that the bottom-most edge of the LEDs 702a, 702b are positioned lower than the bottom-most edge of the camera sensor 701 positions the LEDs 702a, 702b closer to any tissue that is positioned directly in front of the working channel 31, thereby ensuring that such tissue is optimally lit by the pair of LEDs 702a, 702b during a surgical procedure.

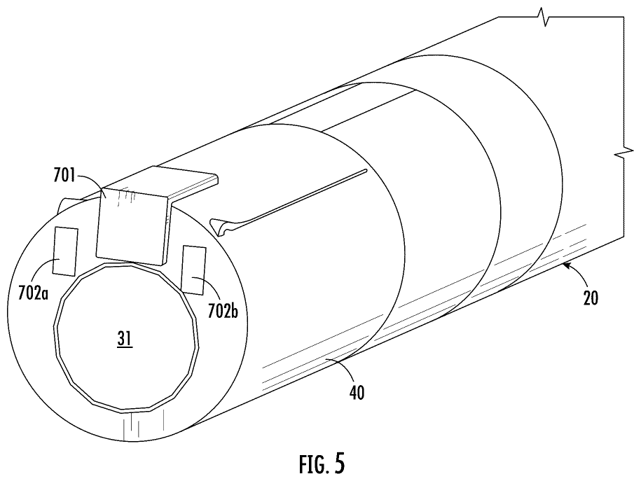

FIG. 5 is a front perspective view similar to that shown in FIG. 4, but having the distal region 40 of the endoscope shaft 20 enclosed by a protective layer such as a micro-molded tip component, as is described further hereinbelow. In various embodiments, the printed circuit board 700 may be mounted to the distal region 40 of the endoscope 20 via one or more of a pattern of braided filaments, e.g., the filaments being alternatingly woven over and under the printed circuit board 70 and around the working channel 31 so as to fix the printed circuit board 700 in position at the distal end of the endoscope 20. Additionally or alternatively, the printed circuit board 700 may be mounted to the distal region 40 of the endoscope 20, via a laminate layer of flexible polymer, such as Pebax® 35 (Pebax® being a tradename for a thermoplastic elastomer of polyether block amide, obtained by, e.g. polycondensation of a carboxylic acid polyamide with an alcohol termination polyether, available commercially from, e.g., Compounding Solutions in Lewiston, ME) or other suitable material. Various possible configurations for the endoscope shaft 20 being formed of a braided pattern of woven filaments and/or having protective laminate layers of flexible polymer are shown and described in additional detail in the above-referenced Applicant's co-pending patent applications. As shown in FIG. 5, neither the pair of LEDs 702a, 702b nor the camera sensor 701 impedes the distal opening into the working channel 31, thereby ensuring an obstruction-free passage of instruments therethrough.

In embodiments, the distalmost tip of the endoscope shaft 20 may be comprised of a micro-molded Pebax® 55D tip component that has an opening sized to accept the camera sensor 701. Such a micro-molded tip component may also include translucent pockets to the sides of the opening that situate the LEDs 702a, 702b adjacent to the camera sensor 701. Having the camera sensor 701 exposed through an opening in such a micro-molded tip component may, according to embodiments and as previously mentioned above, provide an arrangement in which material is not disposed in front of, e.g., distally relative to, the camera sensor 701, thereby preventing or reducing light filtration or distortion. In such an embodiment having pockets therein, the LEDs 702a, 702b may be maintained essentially flush with, or in some embodiments slightly proximal to (as shown in FIG. 3B), a face of the camera sensor 701 to prevent light from, e.g., bleeding, into the camera sensor 701. Still further, the distalmost edges of such a micro-molded tip may be contoured, or otherwise curved, such that the distalmost edges present an atraumatic surface to the patient during insertion and manipulation.

As shown and mentioned above in connection with FIG. 4, the proximal end of the printed circuit board 700 may abut, or otherwise be adjacent to, a distal end of the electrical cable 301 extending longitudinally through the endoscope shaft 20 such that the pair of LEDs 702a, 702b and the camera sensor 701 may each be connected to, e.g., by soldering wires that extend to the distal end of the electrical cable 301. FIG. 6 is a perspective, cut-away view of the handle 50 that illustrates a different portion of the electrical cable 301, specifically a portion of the electrical cable 301 as it extends into the handle 50. FIG. 6 illustrates that, in accordance with embodiments, the electrical cable 301 extends longitudinally through the endoscope shaft 20 so as to be parallel to the working channel 31. As shown, the electrical cable 301 is embedded in, or otherwise disposed within, the wall of the endoscope shaft 20, such that it is disposed laterally outside of the inner diameter of the working channel 31 but laterally inside of the outer diameter of the endoscope shaft 20. The electrical cable 301 emerges from the shaft wall of the endoscope shaft 20 into the handle 50, where it extends proximally to an electronics control module 54 located on a proximal portion of the handle 50 (shown in FIG. 8).

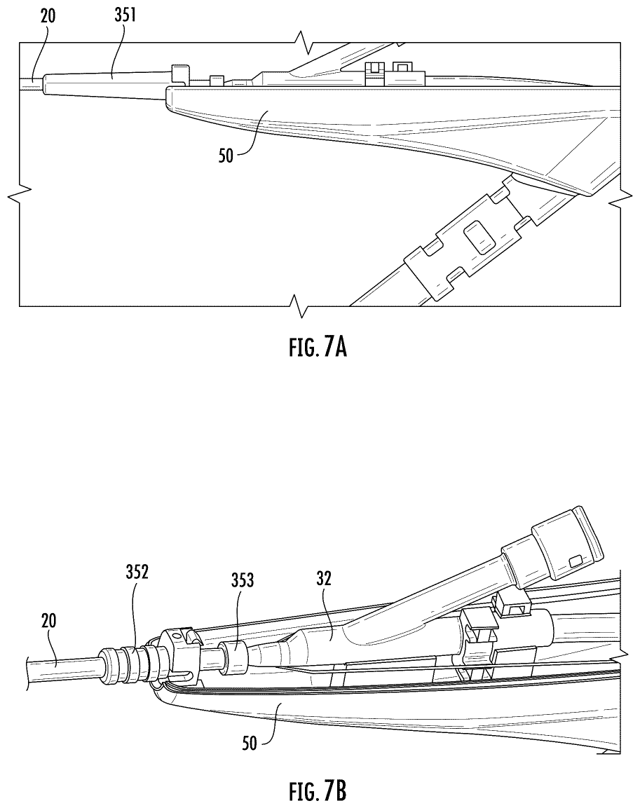

FIGS. 7A and 7B are side and top perspective views (with portions of the handle 50 removed so as not to obscure certain components) that illustrate the shaft 20 connecting to the handle 50, according to an embodiment. FIG. 7A shows a strain relief mechanism 351 extending through the distalmost opening of the handle 50. In an embodiment, the strain relief mechanism 351 is disposed around a portion as the shaft 20 that is within the interior of the handle 50, as well as around a portion of the shaft 20 that extends outside of the handle 50. In this way, the strain relief mechanism 351 may provide some additional reinforcing material that keeps the shaft 20 from overly stressing, and potentially breaking, the material of the handle 50 at the location where the shaft 20 exits the handle 50. As shown in FIG. 7B, the strain relief mechanism 351 may be maintained in position by a ribbed member 352 that grips the strain relief mechanism 351 and prevents relative longitudinal movement thereof. FIG. 7B also illustrates a ring member 353 positioned within the handle 50 and which functions to connect the shaft 20 to the bifurcation region 32. The ring member 353 may also function to maintain the various cables and wires of the shaft 20, e.g., the pull wires and/or the electrical cable 301, in position such that they can thereafter extend proximally to their respective connection points within the handle, and helps provide a simplified structure for connecting to the bifurcation region 32, thereby reducing, e.g., the likelihood of inserted instruments becoming stuck as it passes through the region.

The electronics control module 54 located on the handle 50 of the trans-nasal endoscope 10 may, in accordance with various embodiments, provide additional functionality that may be useful in a surgical procedure being performed thereby. FIG. 8. is a perspective view (with various components, such as the walls of the handle 50, being hidden so as not to obscure the features shown) of a self-contained button assembly 602. The self-contained button assembly 602 may include, among other components, the electronics control module 54 having first and second electrical control buttons 541 and 542. Likewise, the self-contained button assembly 602 may also include the AWS control module 52 having air, water and/or suction buttons 521, 522, 523 disposed thereon, each having respective valves and inlet/outlet openings that enable fluids to be selectively introduced into the working channel 31 of the shaft 20. FIG. 8 illustrates the electronics control module 54 as an electrical button sub-assembly mounted on a circuit board 543. Advantageously, the entire self-contained button assembly 602 is mounted within the handle 50 via a chassis 601. The self-contained button assembly 6902 provides certain advantages, e.g., it enables the button assembly to be functionally independent from the handle 50, thereby allowing it to be assembled, tested, etc., separately from the handle 50 and thereby be potentially manufactured at lower costs.

As mentioned above in connection with FIGS. 1 and 2, the trans-nasal endoscope 10 may also include a video display output cable 57 that extends from the handle 50. FIG. 9 is a schematic view of portions of a video display system, according to various embodiments. As shown in FIG. 9, the proximal end of the video display output cable 57 is connectable to an external video control unit 571. The external video control unit 571 is, in turn, connectable to a video display device 572 configured to display images or video to a user.

Referring to FIGS. 8 and 9, mounted on the self-contained button assembly 602 are various buttons that allow a user to selectively control certain aspects, e.g., the image and/video components, of the trans-nasal endoscope 10. In an embodiment, the external video control unit 571 may include a menu of different video control functions, and the external video control unit 571 may be pre-programmed such that the electrical control buttons 541, 542 are mapped to pre-selected functions within that menu. For example, the first electrical control button 541 may provide a first signal that is mapped to a first function from the menu of video control functions so that the external video control unit 571 causes the first function to be performed when the first electrical control button 541 is pressed, and the second electrical control button 542 may provide a second signal that is mapped to a second function from the menu of video control functions so that the external video control unit 571 causes the second function to be performed when the second electrical control button 542 is pressed.

The menu of video control functions that may be provided by the external video control unit 571, and to which the first and second electrical control buttons may be mapped, may include any one or more of a white balancing function, an image capture function, a zoom function, a video capture start function, a video capture stop function, a brightness change function, a start video conference function, a change color spectrum function, a zoom picture function, a magnify picture function, a measure size of object function, a measure distance of object function, and a launch program or any other keyboard function on an external computer.

Of course, it should be recognized that the electronic control module 54 may have configurations and/or control buttons that are different from those described herein, and that such different configurations and/or control buttons may be employed to control other aspects of an image or video system than described herein. It should also be recognized that certain aspects of the system may employ wireless connections, instead of the wired connections shown herein, to transmit signals and/or data related to the surgical procedure. For example, in embodiments, instead of the video display output cable 57, the trans-nasal endoscope 10 may employ wireless transmitters and receivers located in, e.g., the handle 50 and/or the external video control unit 571, transmit and receive the white balancing control signals and/or the image capture signals.

In operation, when the proximal end of the video display output cable 57 is connected to the external video control unit 571, the external video control unit 571 sends power through the video display output cable 57, through the self-contained button assembly 602, and to the illumination source 42, e.g., the pair of LEDs 702a, 702b shown in FIGS. 3-5, so as to enable the illumination source 42 to provide light at the distal end 40 of the endoscope shaft 20. Likewise, when the proximal end of the video display output cable 57 is connected to the external video control unit 571, the external video control unit 571 sends power through the video display output cable 57, through the self-contained button assembly 602, and to the image capture device 44, e.g., the camera sensor 701 shown in FIGS. 3-5, so as to enable the image capture device 44 to generate image signals at the distal end 40 of the endoscope shaft 20. The image capture device 44 is then configured to send image signals, e.g., related to the tissue located in front of the distal end of the endoscope shaft 20 as lit up by the illumination source 42, back through the self-contained button assembly 602, through the video display output cable 57, and to the external video control unit 571.

These image signals, e.g., related to the tissue located in front of the distal end of the endoscope shaft 20, are received by the external video control unit 571 (via the video display output cable 57), which provides them to the video display device 572 for viewing by a user. In this way, according to various embodiments, a user can view on the video display device 572 a continuous video image of, e.g., the tissue located in front of the distal end of the endoscope shaft 20. This continuous video image may enable a user to more safely insert the distal end of the endoscope shaft 20 into a patient. In addition, this continuous video image may enable a user to continuously view a procedure, e.g., a biopsy procedure performed by a biopsy forceps inserted through the instrument insertion port 30, so as to ensure that it proceeds safely and effectively.

There are no limitations in terms of the particular embodiments described in this application, which are intended as illustrations of various aspects only. Many modifications and variations can be made without departing from its spirit and scope, as will be apparent to those skilled in the art. Functionally equivalent methods and apparatuses, in addition to those enumerated herein, will be apparent to those skilled in the art from the foregoing descriptions. Such modifications and variations are intended to fall within the scope of the appended claims. Only the terms of the appended claims are intended to be limiting, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

1. An endoscope for use in a surgical procedure, comprising:
a handle for gripping by a user;
a shaft extending from the handle, the shaft having a working channel extending longitudinally therethrough, the shaft having a distal region configured to be inserted into a patient;
a camera sensor mounted at the distal region of the shaft, the camera sensor configured for generating image signals related to the surgical procedure;
a self-contained button assembly mounted to the handle, the self-contained button assembly including an electrical button sub-assembly having first and second electrical buttons mounted on a circuit board, the first button configured upon being pressed to cause a first function to be performed relative to the image signals, and a second button configured upon being pressed to cause a second function to be performed relative to the image signals.

2. The endoscope of claim 1, wherein the self-contained button assembly also includes fluid control buttons for controlling the flow of one or more fluids through the working channel.

3. The endoscope of claim 2, wherein the self-contained button assembly is mounted within the handle on a chassis which houses first, second and third fluid valves actuatable by the fluid control buttons.

4. The endoscope of claim 1, wherein the first button is configured to transmit a first signal upon being pressed, and a second button is configured to transmit a second signal upon being pressed.

5. The endoscope of claim 4, further comprising:
a video control unit, the video control unit including a menu of different video control functions, wherein the video control unit is pre-programmed such that the first signal is mapped to the first function from the menu of video control functions so that the video control unit causes the first function to be performed when the first button is pressed, and such that the second signal is mapped to the second function from the menu of video control functions so that the video control unit causes the second function to be performed when the second button is pressed.

6. The endoscope of claim 5, further comprising:
a video display device connected to the video control unit and configured to provide a display corresponding to the image signals generated by the camera sensor.

7. The endoscope of claim 6, wherein the video control unit causes the first or second functions to be performed by the video display device, such that the video display device changes at least one aspect of the display based on the first or second function.

8. The endoscope of claim 5, wherein the menu of video control functions includes one or more of a white balancing function, an image capture function, a zoom function, a video capture start function, a video capture stop function, a brightness change function, a start video conference function, a change color spectrum function, a zoom picture function, a magnify picture function, a measure size of object function, a measure distance of object function, and a launch program or any other keyboard function on an external computer.

9. The endoscope of claim 1, further comprising:
an illumination source at the distal region of the shaft; and
an electrical cable extending longitudinally through the shaft from the illumination source and the camera at the distal region of the shaft circuit board to the handle.

10. The endoscope of claim 1, wherein the surgical procedure is a trans-nasal endoscopy procedure.

11. A self-contained button assembly for use in an endoscope, the endoscope being configured to generate image signals for providing an image corresponding to a surgical procedure, the self-contained button assembly comprising:
first and second electrical buttons mounted on a circuit board, wherein the first electrical button is configured upon being pressed to transmit a first control signal for controlling an aspect of the image, and the second electrical button is configured upon being pressed to transmit a second control signal for controlling an aspect of the image; and
one or more fluid control buttons for controlling the flow of one or more fluids through the endoscope.

12. The self-contained button assembly of claim 11, wherein the first and second electrical buttons are connectable to an image control unit without soldering.

13. The self-contained button assembly of claim 11, wherein the first and second electrical buttons are connectable to an image control unit, the image control unit including at least first and second image control functions, wherein the first control signal is configured to cause the first image control function to be performed, and the second control signal is configured to cause the second image control function to be performed.

14. The self-contained button assembly of claim 13, wherein the first and second image control functions are each one image control function of a menu of different image control functions that are stored within the image control unit, and wherein the first and second control signals are mapped to the first and second image control functions during pre-programming of the image control unit.

15. The self-contained button assembly of claim 14, wherein the first and second control signals are mapped to a menu that includes one or more of a white balancing function, an image capture function, a zoom function, a video capture start function, a video capture stop function, a brightness change function, a start video conference function, a change color spectrum function, a zoom picture function, a magnify picture function, a measure size of object function, a measure distance of object function, and a launch program or any other keyboard function on an external computer.

16. The self-contained button assembly of claim 11, wherein the self-contained button assembly is configured to transmit power from an image control unit to an illumination device located at a distal end of a shaft of the endoscope.

17. The self-contained button assembly of claim 11, wherein the self-contained button assembly is configured to transmit power from an image control unit to a camera device located at a distal end of a shaft of the endoscope, the camera device configured to generate the image signals corresponding to the surgical procedure.

18. The self-contained button assembly of claim 17, wherein the self-contained button assembly is configured to transmit the image signals from the camera device to the image control unit.

19. The self-contained button assembly of claim 18, wherein the image signals transmitted via the self-contained button assembly are processable by the image control unit to generate the image on an image display device.

20-23. (canceled).

24. An endoscope for use in a surgical procedure, comprising:
a handle for gripping by a user;
a shaft extending from the handle, the shaft having a distal region configured to be inserted into a patient;
a micro-molded tip component disposed at the distal region of the shaft, the micro-molded tip component defining at least a first and a second opening; and
a circuit board disposed within the micro-molded tip component, the circuit board including a camera device configured to fit within the first opening of the micro-molded tip component, and the circuit board also including an illumination device configured to fit within the second opening of the micro-molded tip component.

25-30. (canceled).
